(19) United States
(12) Patent Application Publication
(10) Pub. No.: US 2023/0253076 A1
(43) Pub. Date: Aug. 10, 2023

(54) LOCAL STEPS IN LATENT SPACE AND DESCRIPTORS-BASED MOLECULES FILTERING FOR CONDITIONAL MOLECULAR GENERATION

(71) Applicant: Insilico Medicine IP Limited, Hong Kong (HK)

(72) Inventors: Iskander Safiulin, Saint Petersburg (RU); Evgeny Olegovich Putin, St. Petersburg (RU); Aleksandrs Zavoronkovs, Pak Shek Kok (HK)

(21) Appl. No.: 18/164,160

(22) Filed: Feb. 3, 2023

Related U.S. Application Data
(60) Provisional application No. 63/267,660, filed on Feb. 7, 2022.

(51) Int. Cl. G16C 20/50; G16C 20/30; G16C 20/70; G06N 3/0455; G06N 3/0475; G06N 3/088

(52) U.S. Cl. CPC G16C 20/50; G16C 20/30; G16C 20/70; G06N 3/0455; G06N 3/0475; G06N 3/088

(57) ABSTRACT

A method of generating molecular structures includes: providing an ABGM; inputting into the ABGM scored molecules having an objective function value; selecting scored molecules with large objective function values; processing the selected scored molecules through an encoder to obtain latent points; selecting a latent point; sampling neighbor latent points that are within a distance from the selected latent point; processing the sampled neighbor latent points with a decoder to generate generated molecules; and provide a report having at least one generated molecule. The scored molecules can have at least one desired property. The method can include: comparing the generated molecules with selected scored molecules; selecting molecules from the generated molecules that are closest to the selected scored molecules; and providing the selected molecules as candidates for having the at least one property.

### CROSS-REFERENCE TO RELATED APPLICATIONS

This patent application claims priority to U.S. Provisional Application No. 63/267,660 filed Feb. 7, 2022, which provisional is incorporated herein by specific reference in its entirety.

### BACKGROUND

### Field

The present disclosure relates to computer-implemented protocols to generate molecules that have one or more desired properties.

### Description of Related Art

Previously, drug design, often referred to as rational drug design or simply rational design, has been the process of finding new medications based on the knowledge of a biological target. The drug can be an organic small molecule that activates or inhibits the function of a biomolecule, such as a protein, which in turn results in a therapeutic benefit to the patient. In the most basic sense, drug design involves the design of molecules that are complementary in shape and charge to the biomolecular target with which they interact and thereby will bind to it. Drug design that relies on the knowledge of the three-dimensional structure of the biomolecular target is known as structure-based drug design.

Artificial Neural Networks (ANNs) are computing systems inspired by the biological neural networks that constitute animal brains. An ANN is based on a collection of connected units or nodes called artificial neurons, which loosely model the neurons in a biological brain. Each connection, like the synapses in a biological brain, can transmit a signal to other neurons. An artificial neuron receives a signal then processes it and can signal neurons connected to it. The “signal” at a connection is a real number, and the output of each neuron is computed by some non-linear function of the sum of its inputs. The connections are called edges. Neurons and edges typically have a weight that adjusts as learning proceeds. The weight increases or decreases the strength of the signal at a connection. Neurons may have a threshold such that a signal is sent only when the aggregate signal crosses that threshold. Typically, neurons are aggregated into layers. Different layers may perform different transformations on their inputs. Signals travel from the first layer (the input layer) to the last layer (the output layer), possibly after traversing the layers multiple times.

Deep Neural Networks (DNNs) are ANNs with one or more hidden layers. These networks, due to their complex structure and a large number of trainable parameters, make it possible to solve problems more efficiently. Autoencoders are a subset of DNNs that learn the hidden representation of objects. Objects can be different mathematically formalized objects, for example—strings, graphs, or pictures. An autoencoder includes two parts—an encoder and a decoder. An encoder is an encoding function that maps an object to a point (e.g., latent point) in a numerical space with a specified dimension. This numerical space is called latent space. A decoder is a decoding function that maps a point in latent space to an object in the object space. For training, these networks use reconstruction loss, a function that penalizes the model for differences between the input (encoder input) and output (decoder output) representations of an object.

Generative models (GM) are a subclass of DNNs that enable the generation of objects. Unlike standard DNNs that predict the properties of objects, these networks are trained in such a way as to generate new objects in the future without input data. These models learn the distribution of objects (e.g., distributional learning) and then try to generate samples from this distribution.

Autoencoder-based generative models (ABGM) are generative models that are based on autoencoder architecture. For the generating process, these models use different mechanics for learning and interacting with the latent space. The most popular representatives of this class of models are Adversarial Autoencoder (AAE) and Variational Autoencoder (VAE). Both of these networks use different learning techniques, the goal of which is to ensure that the distribution of representations of objects in the latent space is as close as possible to some given distribution, such as normal (normal distribution). If the network is trained well, then the generation process will be to randomly sample points from this given distribution and decode them using a decoder part of the model. Another type of generative model is the Generative Adversarial Network (GAN), which is a network that uses a latent space for sampling molecules, but it is not an autoencoder-based generative model since it does not have an encoder part of the network. This model uses the mechanism of an adversarial game for learning latent space distribution.

Distributional learning generative models generate random molecules by default. However, sometimes one wants to generate objects that satisfy given properties. This formulation of the problem is called conditional generation.

In recent years, DNNs have been actively used to solve the problem of drug design. For example, generative models can create molecules that satisfy the conditions in the drug design problem. These generative models use different versions of the mathematical representation of molecules. One of the most popular of these representations of molecules is SMILES [1], which is in the form of a chemical line notation for describing the structure of chemical species using short ASCII strings. Another type of representation of molecules is graph. Mathematically, a molecule graph can be represented in many ways, one of the most popular being the adjacency matrix.

A Recurrent Neural Network (RNN) is a type of neural network that contains loops, which allows information to be stored within the network. RNN uses their reasoning from previous experiences to inform the upcoming events. Recurrent models are usually used for tasks related to the textual representation of input data, such as, for example, SMILES representation of molecules. The Long Short Term Memory Network (LSTM) is an advanced RNN, which is a sequential network that allows information to persist. It is capable of handling the vanishing gradient problem that can be faced by an RNN.

### SUMMARY

In some embodiments, a method of generating molecular structures is provided. The method can include providing an autoencoder-based generative model (ABGM) for generation of molecular structures. The database of scored molecules can be input into the autoencoder-based generative model. Each scored molecule can have an objective function value that is calculated from an objective function. The scored molecules can be selected from the database to have relatively larger objective function values compared to other scored molecules in the database. The selected scored molecules can be processed through an encoder of the autoencoder-based generative model to obtain latent points in a latent space. A latent point in the latent space can be selected, and neighbor latent points can be sampled that are within a distance from the selected latent point. The sampled neighbor latent points can be processed with a decoder to generate at least one generated molecule. A report having the at least one generated molecule can be provided. In some aspects, the scored molecules have at least one property. In some aspects, the method can include comparing the generated molecules with the selected scored molecules and selecting molecules from the generated molecules that are closest to the selected scored molecules. The selected molecules can be provided as candidates for having the at least one property.

In some embodiments, the methods can include steps of selecting certain generated molecules. The selecting can be based on at least one of a fingerprint molecule clustering and sampling protocol; and/or an acceptance function having an acceptance function value equal to 1.

In some embodiments, a fingerprint molecule clustering and sampling protocol can be performed by selecting scored molecules from the database that have the acceptance function value equal to 1. Fingerprints can be calculated for the selected scored molecules. The selected scored molecules can be selected based on the fingerprint vector. The top number of molecules in each cluster can be selected. The selected top number of molecules can be sorted by objective function value. Then, the method can randomly sample one molecule from each cluster; and provide the randomly sampled molecule from each cluster in the report. In some aspects, the fingerprint is a Morgan fingerprint, extended connectivity fingerprint (ECFP), or other molecular fingerprint.

In some embodiments, a method of obtaining local latent spaces can be performed by a local steps in latent space protocol. The method can include determining a latent point as a starting point and then determining a step length, number of levels, and number of steps in each level. When a number of latent points in a sampled points list is less than a threshold, the following local steps in latent space protocol can be performed: (a) sample a number of random points in the latent space; (b) sample neighboring points within a defined distance from the sampled random points; (c) add the sampled neighboring points to the sample points list; (d) increase the defined distance; and Repeat steps (a)-(d) until the number of latent points in the sampled points list is equal to the threshold, and then provide the sample points list having the threshold number of latent points.

In some embodiments, a method of selecting generated molecules can be provided. The method can include training the ABGM with the scored molecules. Scored molecules with high objective function value that are diverse can be selected to obtain encodable molecules. The encodable molecules can be encoded into latent points in the latent space using the encoder. New latent points in the latent space can be obtained that are neighboring latent points to the selected latent points. The new latent points can be decoded into newly generated molecules using the decoder. An objective function value can be calculated for the newly generated molecules. The database of molecules can be updated to include the newly generated molecules with the calculated objective function value. In some aspects, the method can include filtering the newly generated molecules for valid molecules. In some aspects, the method can include selecting newly generated molecules that are closest in latent space to each other. In some aspects, the newly generated molecules are selected by: determine a property for a target molecule; obtain a potential set of molecules; determine a similarity metric for the molecules in the potential set; and select molecules in the potential set with the similarity metric that is closest to the target molecule having the property.

In some embodiments, molecular descriptors are used for selecting molecules. The method can include: calculating molecular descriptors of the generated molecules; calculating molecular descriptors of the selected molecules; comparing molecular descriptors of the generated molecules to molecular descriptors of the selected molecules; selecting generated molecules with molecular descriptors closest to target molecules; and providing the selected generated molecules that are closes to target molecules.

In some embodiments, good and diverse molecules are selected. These molecules can be good by having the higher objective function value and can be diverse by being picked from different groupings of molecules. The molecules can be selected by selecting the target molecules by a protocol that selects diverse molecules, which can be molecules with high objective function with diverse structural characteristics. The protocol that selects the diverse molecules can include selecting scored molecules from the database that have an acceptance function value equal to 1. Then, fingerprints can be calculated for the selected scored molecules, where the fingerprints can include a fingerprint vector. The selected scored molecules can be clustered into different clusters by the fingerprint vector, where similar fingerprint vectors are grouped together, thereby forming multiple clustered groups. The top number of molecules in each cluster can be selected and sorted by objective function value. From these selected top numbers of molecules from each cluster, there can be a random sampling of one molecule from each cluster. The randomly sampled molecule from each cluster can be provided in the report.

In some embodiments, molecular descriptors are used for selecting generated molecules that have a desired property. The method can include calculating molecular descriptors as one or more of the following: number of hydrogen bond acceptors; number of hydrogen bond donors; partition coefficient of a molecule between aqueous and lipophilic phases; a topological polar surface area; a zagreb index of molecule; and an electro topological index. In some aspects, a similarity metric can be used to select molecules, which similarity metric can be based on the molecular descriptors. The method can include: calculating similarity metric between molecules based on the molecular descriptors; and selecting generated molecules closest to similarity metric.

In some embodiments, the selection of generated molecules can include: select acceptable molecules with AF(x)=1; calculate a chemical fingerprint for selected molecules; apply the clustering method on the calculated fingerprints; select in every cluster N molecules with highest values of objective function; and from the selected molecules, randomly choose one molecule in every cluster.

In some embodiments, the selection of generated molecules can include: selecting molecules with an acceptance function of 1; calculating chemical fingerprints for each selected molecule; clustering molecules by fingerprint vector; selecting top molecules in each cluster; sorting molecules by objective function; and selecting molecules with relatively higher objective function in each cluster or randomly sample one molecule in each cluster.

In some embodiments, a method of selecting molecules with at least one desired property can be provided. The method can include generating generated molecules with the generative model. A base of scored molecules can be provided, which have the objective function value as the score. A selection of molecules can be performed to obtain different molecules with high scores from the base. The generated molecules and the selected molecules can be compared for selecting generated molecules closest to a high score of the selected molecules. The selected generated molecules can be identified as candidates to have at least one defined property.

In some embodiments, a method of building a database of molecules with calculated objective function values can be provided. The method can include training the ABGM with molecules having the high objective function value. Molecules can be selected with a procedure that selects molecules that have high objective function value and diverse in structure, which can be referred to as a good and diverse molecules selection protocol. Then, molecules can be encoded to latent points using encoder. New latent points in the latent space can be created using a protocol, which can be referred to as a latent space making step protocol. The new latent points can be decoded into new generated molecules using the decoder. New and valid generated molecules can be filtered for. The newly generated molecules that are determined to be molecules that are closest in the latent space can be selected. The objective function can be calculated for each of these newly generated molecules, which can be added as generated molecules to the database having molecules with calculated objective function values.

In some embodiments, a method of selecting similar molecules can be provided. The method can include obtaining a batch of candidate molecules from the generated molecules and calculating a descriptor vector for each candidate molecule. Diverse molecules can be selected from a cluster of molecules that are sorted by objective function value. The descriptor vectors for selected diverse molecules can be calculated. A similarity metric can be calculated between molecules based on the molecular descriptors, and generated molecules that are closest to the similarity metric can be calculated.

In some embodiments, one or more non-transitory computer readable media are provided that store instructions that in response to being executed by one or more processors, cause a computer system to perform operations, the operations comprising the method of one of the embodiments recited herein.

In some embodiments, a computer system can include: one or more processors; and one or more non-transitory computer readable media storing instructions that in response to being executed by the one or more processors, cause the computer system to perform operations, the operations comprising the method of one of the embodiments.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates a scheme of autoencoder-based generative model architecture.

FIG. 2 illustrates an overall scheme of a local steps in latent space (LSLS) molecular generation protocol.

FIG. 3 illustrates an overall scheme of a descriptors-based molecules filtration protocol.

FIG. 4 illustrates a scheme of a fingerprints-based clustering and sampling protocol.

FIG. 5 illustrates a scheme of a latent neighbor sampling protocol.

FIG. 6 illustrates a scheme of a local steps in latent space (LSLS) generation protocol.

FIG. 7 illustrates a scheme of a similar molecules selection protocol.

FIG. 8 illustrates a scheme of a descriptors-based molecules filtration protocol.

FIG. 9 illustrates data of molecule weight of generated molecules.

FIG. 10 illustrates data of number of atoms of generated molecules.

FIG. 11 illustrates an example of a computer that can be used in the computing systems described herein to perform the computer-implemented methods.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

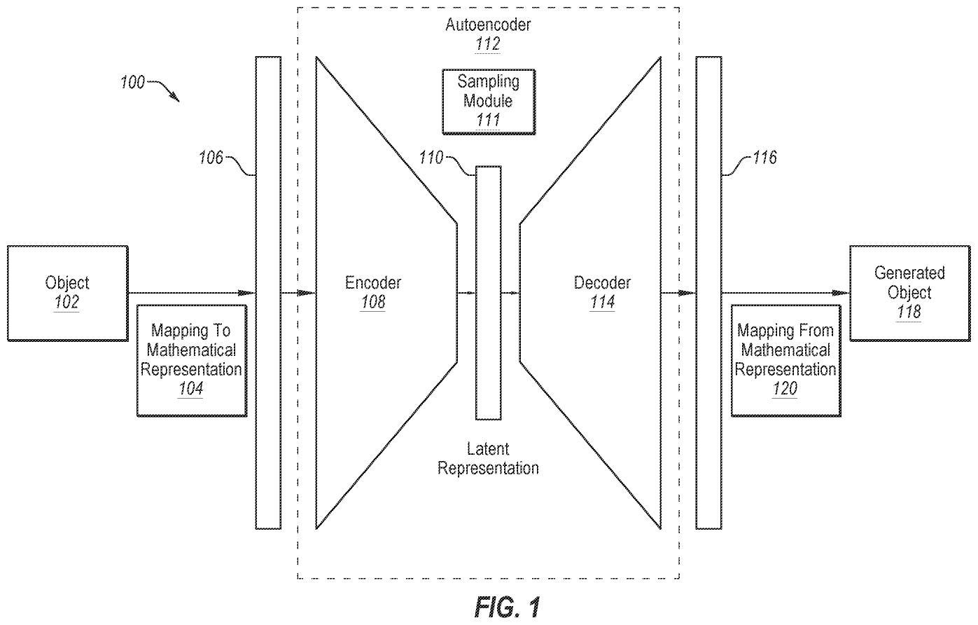

Generally, the present technology can utilize an autoencoder-based generative model (ABGM) architecture for chemical structure design. The ABGM architecture 100 is shown in FIG. 1 and described below. The ABGM architecture 100 can include object data 102 that is mapped 104 to a mathematical representation of the object 106. The object mathematical representation 106 is then processed by an encoder 108 to obtain the latent representation data in the latent space 110. The encoder 108 is part of the autoencoder 112 of the ABGM architecture 100. The latent representation data of the latent space 110 is then processed through the decoder 114 of the ABGM architecture 100 to obtain the mathematical representation of a generated object 118. The generated object mathematical representation is then processed to obtain the generated object 118 by mapping 120 from the generated object mathematical representation to obtain the structure of the generated object.

The ABGM architecture 100 can be used in drug design in order to generate molecules (i.e., objects) that satisfy some properties (e.g., biological activity for drug function). The properties can be defined such that the generated molecule has one or more defined properties. Each component of these defined properties can be expressed by some mathematical function. Accordingly, the ABGM architecture 100 can be utilized so that the molecule data is processed to generate the generated molecules. The generated molecules are constrained to those that satisfy the properties. In some aspects, the mathematic function of a property can be provided in a form that receives a representation of the final evaluation function of the generated molecule.

In some embodiments, the ABGM architecture 100 can distinguish between two types of such mathematical functions. Firstly, the function that evaluates the quality of the molecule in the context of the task (e.g., required property) can be referred to as the objective function OF: x→R. It has been found that the larger the value of this function, the more suitable the molecule for the task, and thereby has the desired property. Secondly, the function that evaluates whether a molecule (x) is acceptable for the task (e.g., required property) is called the acceptance function—AF: x→{0,1}. If AF(x)=1, then the x molecule is acceptable for the task (e.g., has the required property). Otherwise AF(x)=0 when the x molecule is not suitable for the task because it lacks the required property.

In some embodiments, calculating the OF and AF functions can be a complex process that takes a lot of time, such as because of calculation of complex biochemical properties of a molecule is calculation intensive. Thus, the generative models (e.g., ABGM architecture 100) can be configured to generate molecules with high objective function in order to have the required property. This can avoid wasting time calculating the objective function for low-quality molecules. In this regard, the use of conditional generation protocols becomes especially important for obtaining molecules that have required properties. For example, now drug-design tasks can be performed to obtain a generated molecule that has the required function of the biological activity in order to treat a disease or condition. Therefore, the task can be for generating a molecule that can function as a drug with the required property of biological activity to modulate a biological protein, such as by inhibiting a biological pathway or restoring function of a biological pathway.

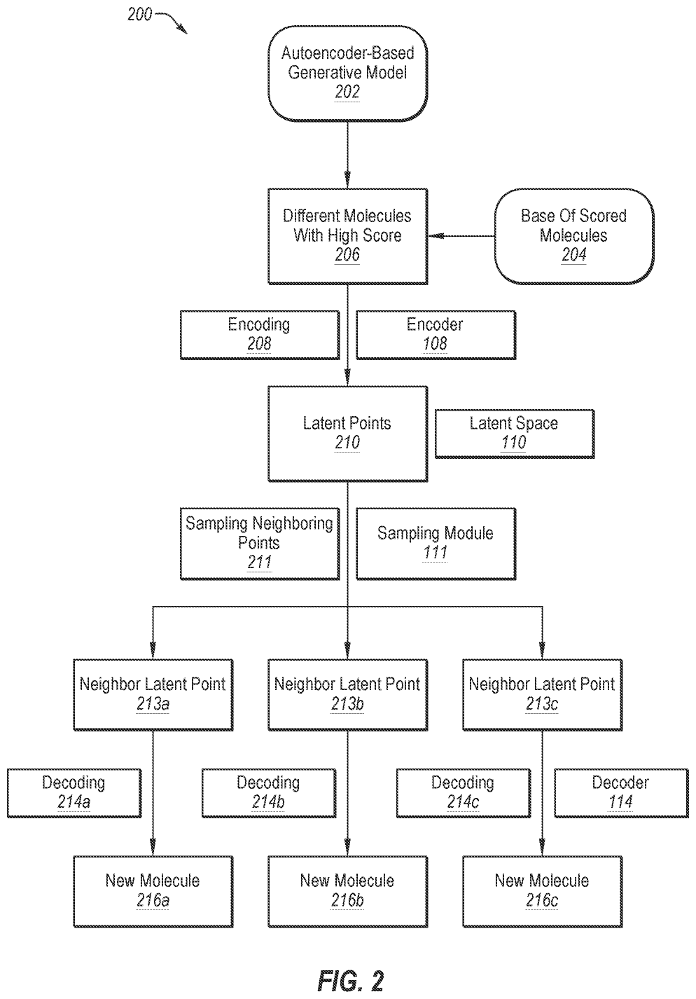

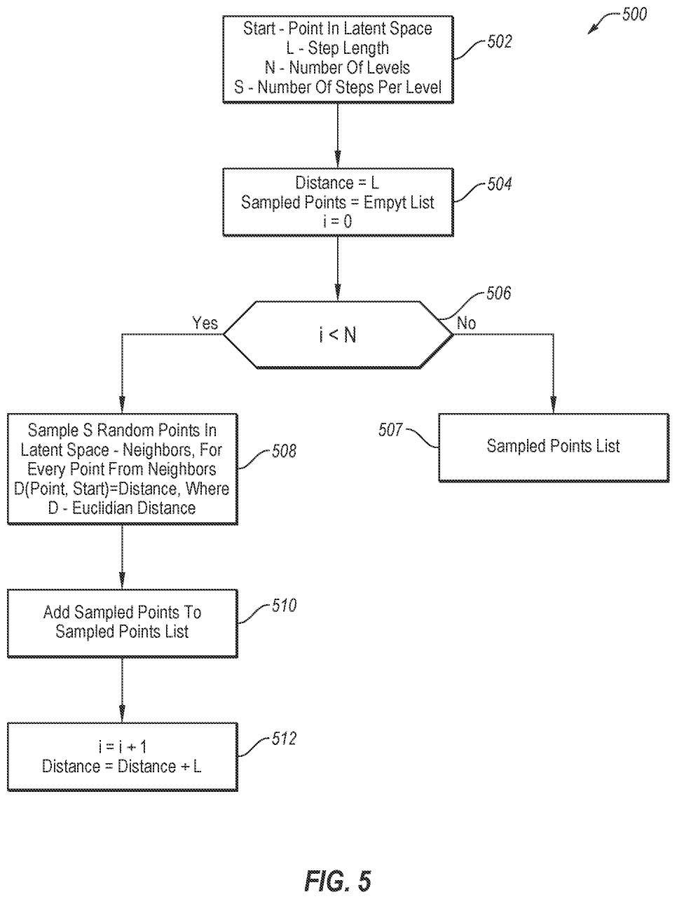

In some embodiments, the ABGM architecture 100 can be used for two conditional generation mechanisms that work with models in a plug and play fashion in order to generate objects that have a property, such as molecules being a drug with a biological activity. In some aspects, a first protocol can be a performed with local steps in the latent space generation (see FIG. 2). This protocol can be applied to any configuration of an autoencoder-based generative model. As used herein, reference to local steps in latent space protocols can refer to FIG. 2. An example of a method of performing the local steps in latent space protocol is shown in FIG. 5.

FIG. 2 shows the local steps in latent space protocol 200 for molecule generation. As shown, an ABGM (e.g., ABGM architecture 100) can be provided to the computing system (block 202), which can be stored on a non-transient tangible memory device. A base of scored molecules is provided to the computing system (block 204), which scored molecules can have low scores, median scores, or high scores. The molecules with the high scores can be selected for processing with the ABGM (block 206). The scores can be based on the molecule having the one or more desired properties. In some aspects, the properties can be ranked from least important to most important, with higher scores being assigned to properties that are more desired.

The selected molecules with high scores are then processed with the AGBM, such as with encoding into latent points 210 (e.g., data) in the latent space 110 with the encoder 108. The latent points 210 are sampled 212 with the sampling module 111, which samples the latent data in the latent space 110. The sampling can be random or based on criteria (e.g., the property), or can be weighted (e.g., higher objective function score). The sampling module 111 can sample neighbor latent points 213a, 213b, 213c, which are neighboring the latent points 210 in the latent space 110. These latent points can be neighbors to each other and/or neighbors to a selected latent point. Each of the neighbor latent points 213a, 213b, 213c are then processed through the decoder 114 for decoding into newly generated molecules 216a, 216b, 216c (e.g., new molecules). Of course, any number of neighbor latent points 213a-c can be sampled within reason. As a result, the new molecules 216a-c have the one or more desired properties and may include the highest ranked priority. The new molecules may have high objective functions and may have the desired property.

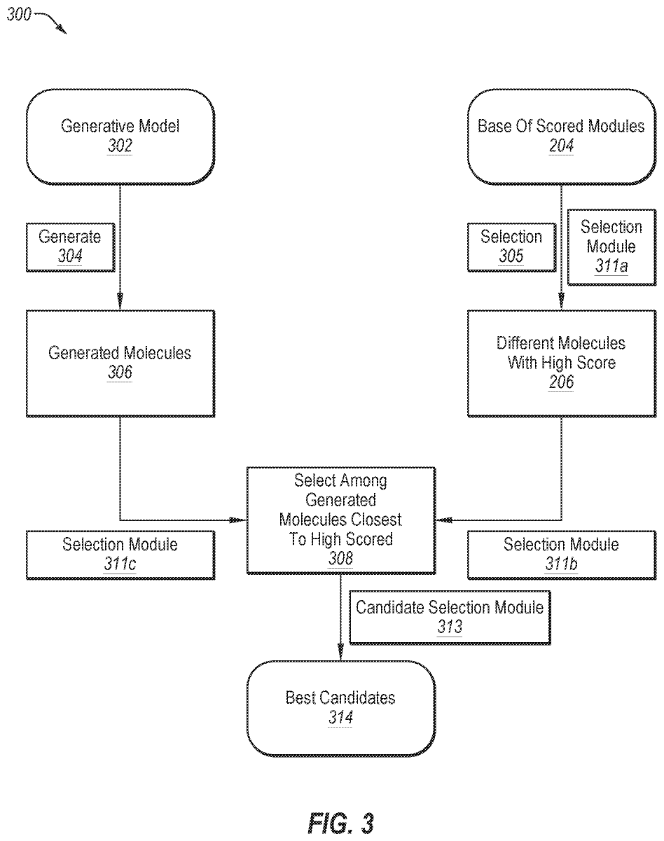

In some aspects, a second protocol can be configured for a descriptors-based filtration of the generated molecules (see FIG. 3). For this protocol there are no conditions for the generative model. The generated molecules are selected based on their molecular descriptors. Therefore, the first protocol of using the latent steps in latent space to obtain generated molecules with a high likelihood of having the desired property can be combined with the second protocol for descriptors-based molecular filtering in order to obtain molecules that have the property. Thus, high quality molecules that have the property and satisfy certain criteria for use can be generated as described herein.

In some embodiments, the local steps in latent space protocol can be used for molecules. The local steps in latent space protocol can function as a latent neighbor sampling protocol, where neighbor latent points are sampled together. This neighbor sampling can be the local steps, which are in the latent space.

FIG. 3 shows the descriptors-based filtration protocol 300 to obtain filtered molecules. The molecules can be filtered to obtain those with the one or more desired properties, such as having the highest ranked property value. FIG. 3 can be reviewed in view of FIG. 2, where the base of scored molecules can be the same. While the generative model of FIG. 3 can be the AGBM, other generative models can be used. As shown, the base of scored molecules 204 (e.g., have objective function value) can be used for a selection process 305 with a selection module 311a that is adapted for selecting the molecules with high scores (e.g., above an objective function value threshold or score threshold), where the selection can be similar as in FIG. 2. As a result, a database of different molecules with high scores 206 (FIG. 2) is obtained. From this database of different molecules with high scores 206, another selection module 311b (or the same selection module with different selection priorities) can be used to select molecules for comparison. A selection protocol based on the molecular descriptors can be used, such as described herein.

A generative model 302 can be used in a generation protocol (e.g., FIG. 2) to generate molecules 306, which can be stored in a database. Another selection module 311c (or the same selection module with different selection priorities) can be used to select generated molecules for comparison with the selected molecules (206). The selection can be performed to obtain generated molecules that are the closest to the selected molecules with the high scores 308. A candidate selection module 313 can be used to select the best candidates to have the one or more desired properties from the generated molecules that are closest to the high scored molecules, which results in the best candidates 314. The protocol of FIG. 3 can be used as a selection protocol for selection of molecules that are the closest to the molecules of the database that have the high scores.

In some embodiments, fingerprint-based molecule clustering and sampling can be performed, which can be a procedure for high-scored and diverse molecule selection. In some aspects, an important part of a drug design task is creating different molecules to analyze for having the property; however, this can apply to other objects with desired properties. For proposing generative mechanisms, a procedure for selecting diverse molecules is used. This procedure can be referred to as a fingerprint-based molecule clustering and sampling procedure, which is applicable for a set of molecules with calculated objective and acceptance functions. See the protocols for the objective and acceptance functions.

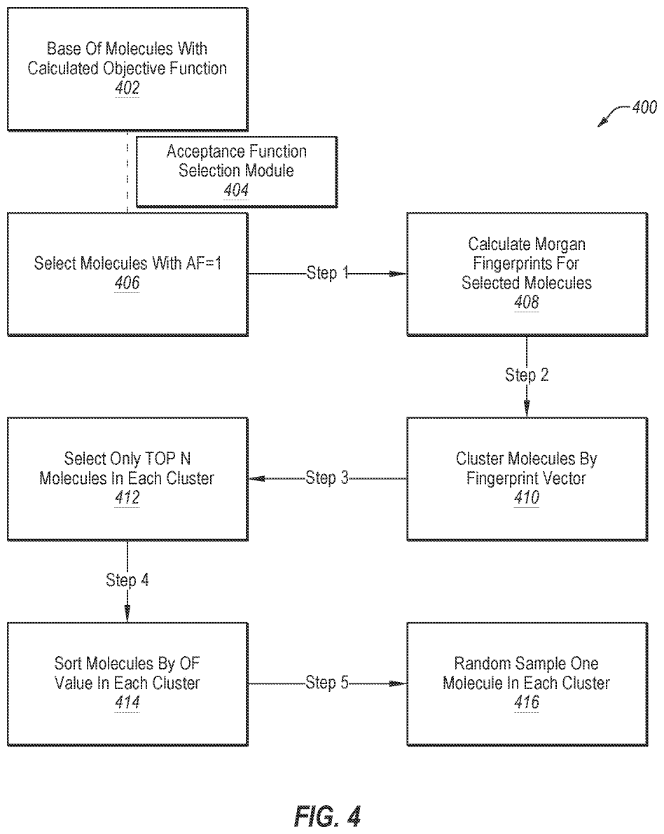

FIG. 4 shows an example of a fingerprint-based molecule clustering and sampling procedure 400. This procedure 400 can be performed with any of the election modules 311a-c or 314 of FIG. 3. This procedure 400 may also be considered to be a diverse molecule selection protocol that selects molecules with high scores, and which provides diverse molecules that can have diverse structures. The procedure 400 can initiate with a database of molecules that have a calculated objection function, such as described herein (block 402), which can be provided to the computing system. Then, an acceptance function selector module 404 can be used to select molecules with the acceptance function being equal to 1 (AF=1), to obtain selected molecules (block 406). Then, the fingerprints (Morgan fingerprints: doi.org/10.1021/c160017a018), or other fingerprints are calculated (block 408) for the selected molecules 406. Then, the selected molecules are clustered into different groups based on the fingerprint vectors of the fingerprints (block 410). Then, the top N (e.g., N is an integer, which are the top or highest scored molecules) molecules of each cluster are selected (block 412). Then, the selected top N molecules are sorted by the objective function value thereof from each cluster (block 414). As a result, each cluster can have the molecules sorted by the objection function value. Then, each cluster is randomly sampled to obtain one randomly sampled molecule in each cluster (block 416). As a result, the sampled molecules are obtained, which have the objective function and acceptance function equal to 1. As a result of procedure 400, diverse molecules that have high objective function are obtained. This procedure may be referred to as the good and diverse molecules selection procedure.

An example protocol of FIG. 4 is as follows: (1) Select acceptable molecules (AF(x)=1); (2) Calculate Morgan fingerprints for selected molecules; (3) Apply the clustering method on the calculated fingerprints; (4) Select in every cluster N molecules with the highest values of objective function; and (5) From the selected molecules, randomly choose one molecule in every cluster. This results in a molecule from each cluster being selected. The selected molecules are a subset of different (in terms of chemistry) molecules with the best objective function values. Therefore, these selected molecules likely have the one or more properties.

In some embodiments, the fingerprint can be a Morgan fingerprint or any other similar machine description for chemical structures. The Morgan fingerprint is basically a reimplementation of the extended connectivity fingerprint (ECFP). In essence, the protocol goes through each atom of the molecule and obtains all possible paths through this atom with a specific radius. Then, each unique path is hashed into a number with a maximum based on bit number. The higher the radius, the bigger fragments are encoded. So, a Morgan radius 2 has all paths found in Morgan radius 1 and then some additional ones. In general, people use radius 2 (similar to ECFP4) and 3 (similar to ECFP6). As for number of bits it depends on the dataset. The higher bit number the more discriminative your fingerprint can be. If you have a large and diverse dataset but only have 32 bits, it will not be good. Start with 1024 bits, but also check higher numbers and see if you are losing too much information. Thus, one of ordinary skill in the art would understand a molecular fingerprint and how to obtain the fingerprint vector.

FIG. 5 illustrates an example of local steps in latent space protocol 500. The protocol 500 is used in the main protocol for generating points in latent space. The protocol 500 can be used with a point from latent space (Start), step length (L), number of levels (N) and number of steps per level (S) (block 502). As shown, the distance is set to equal L, with the Sampled Points list (e.g., Sampled Points, or Sample Points) being equal to an empty list, and with i (e.g., number of latent points in Sample Points list) equal to zero (0) (block 504). Decision block 506 determines whether or not i is less than N. If i is not less than N (No), then it goes to the Sampled Points list, which is complete. If i is less than N (Yes), then it goes to sample S random latent points in the latent space, wherein neighbors for the sampled latent point are a distance away, and every latent point for the neighbors can be a distance, such as the distance being (e.g., d(Point, Start)) equal to the defined distance (e.g., distance is the step length), wherein the distance (d) is the Euclidean distance (block 508). Then, sampled points are added to the Sampled Points list (block 510). Then, i is set to be equal to i+1, and the distance of the neighbors is set to be equal to Distance+L (block 512). Then, the iteration loop is back to decision block 506 which determines whether or not i is less than N. If yes, another iteration loop is performed (block 508, block 510, and block 512). If yes, then the Sampled Points list is provided. With this procedure, a set of points are obtained surrounding the origin of the latent space at different distances.

An example protocol of FIG. 5 is as follows: (1) Distance=L; (2) Sampled Points=empty list; (3) For k in 1; N do the following; (4) Sample S random points in Latent Space—Neighbors are sampled, for every Point from Neighbors d(Point, Start)=Distance, where d=Euclidean distance; (5) Add points to Sampled Points; and (6) Distance=Distance+L. The k can be relative to the property or the initial latent point in the latent space, which would have the property.

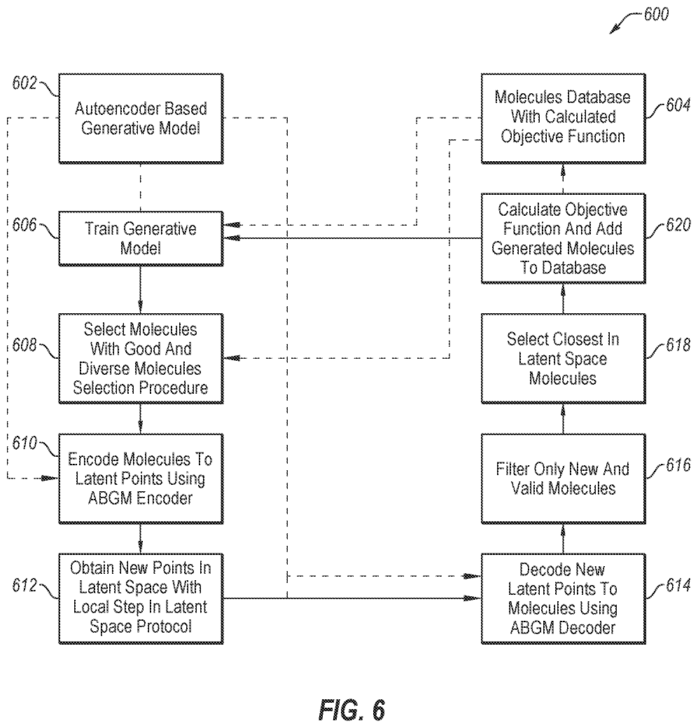

In some embodiments, a main protocol 600 may be utilized as shown in FIG. 6. The main protocol 600 can include providing an ABGM (block 602), such as described herein. Also, the main protocol 600 can include providing a database of molecules having a calculated objective function (block 604), such as described herein. The ABGM can be trained with the molecules with the calculated objective function from the database (bock 606). Then, molecules are selected that have high scores (good) that are diverse (e.g., thereby “good and diverse molecules selection”), which is at block 608. Also, the ABGM encoder is used to encode generated molecules to latent points (block 610). New latent points in the latent space are obtained or created using the local steps in latent space protocol 500 (FIG. 5), as in block 612. Then, obtained new latent points in the latent space are decoded into new generated molecules using the ABGM decoder (block 614). Then, only new molecules that are also valid molecules (e.g., that have the property) are filtered and obtained (block 616). Then, the closest molecules in the latent space are selected (block 618). The objective function of the generated molecules that are selected is calculated, and the generated molecules are added to the database (block 620). Also, the dashed lines show that the ABGM can encode molecules as described herein, which can be obtained from the selected molecules of block 608. Additionally, the dashed lines show that the ABGM can decode the new latent points to obtain the newly generated molecules (block 614) that are then filtered, selected, and have their objective function calculated. Also, the molecules in the database can be selected to be part of the selected molecules. Accordingly, the molecules that are encoded into the latent points of the latent space may be the selected molecule, from the database.

The local steps in latent space molecules generating process (FIG. 5) are applicable for ABGM, which was pretrained on molecules from a distribution similar to the desired molecules (e.g., having the one or more desired properties—high objective function value above a threshold). The database of molecules with calculated objective function values can be used with the ABGM, such as for training, which allows for enhanced ability to obtain generated molecules with the one or more properties. This generative procedure allows using the ABGM to generate new generated molecules similar to already found molecules that have a high value of the objective function (high objective function value).

In some embodiments, the main protocol includes the following steps (see FIG. 6): (1) Train AGBM on data from the database of molecules with the calculated objective function; (2) Select diverse molecules using the “good and diverse molecules selection” protocol (e.g., see FIG. 4) from the base of molecules with a calculated objective function (e.g., these are the target molecules having the property); (3) Encode the target molecules to the AGBM latent space using the AGBM encoder; (4) Create or obtain new latent points in the latent space using the “local steps in latent space” protocol (e.g., see FIG. 5); (5) Decode new latent points to obtain newly generated molecules using the decoder; (6) Filter only new and valid molecules; (7) Select closest to target molecules by Euclidean distance in latent space; (8) Calculate the objective function for the selected generated molecules and add the generated molecules to the database; (9) Return to Step 1. Alternatively, the generated molecules can be selected for validation in order to ensure that they have the desired property. A generated molecule with a desired property can then be obtained as a physical copy (e.g., synthesized) and validated to have the property that is desired. The validation can be a biological assay or a physicochemical assay to characterize the physical copy of the generated molecule and prove it has the desired property.

During the processing of the main protocol, the ABGM learns the probability distribution of molecules, including molecules with high values of objective function. This allows a more accurate representation of these molecules with high objective function value in the latent space of the model. As such, the protocol makes it possible to effectively sample the neighbors of these molecules in the latent space of the model, thereby generating similar molecules. This provides the local steps in the latent space for sampling and molecule generation.

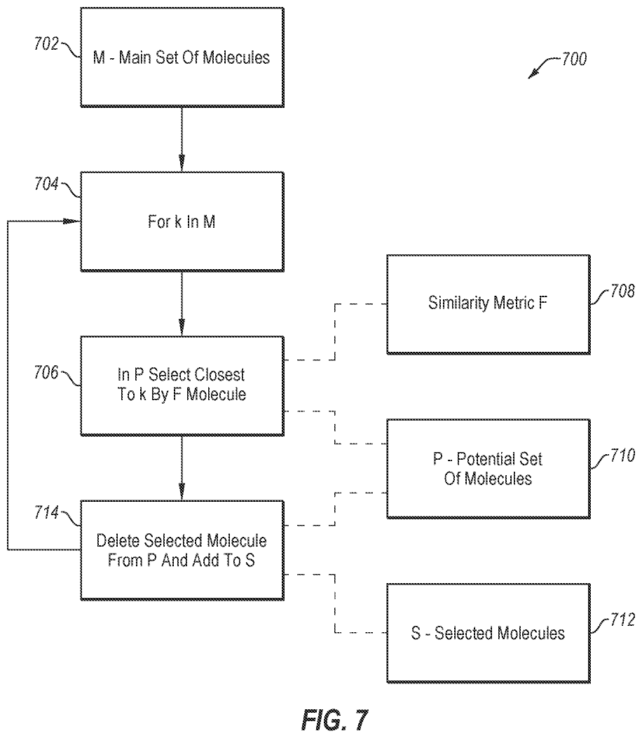

FIG. 7 shows a procedure for selecting similar molecules 700, which can be applied as described herein. As shown, the procedure can include obtaining a main set of molecules M (block 702). A number of the molecules in the main set are selected, which can include the property k in the M main set of molecules (block 704). A potential set of molecules is selected to be the closest to k by the similarity metric F (block 706). The similarity metric can be F(x,y), such as molecules x having the property y. Accordingly, the similarity metric F can be determined for each molecule (708). Therefore, in the potential set, molecules that are closest to k by the similarity metric F are selected (706). Also, the potential set of molecules P can be determined (710), which can be used to select the molecules closest to k. Molecules are selected as selected molecules that have a minimal value of the similarity metric F, which are the S—selected molecules (block 712). The selected molecules are deleted from the potential set of molecules P and added to the selected molecules—S (block 714). The protocol can iterate back to block 704 and run through the protocol again until the S selected molecules is appropriate. This can obtain a group of selected molecules that are similar. Accordingly, this protocol can be used by any selection step for selecting similar molecules, such as in FIG. 3 in the selection modules 311a-c or block 518 in FIG. 6.

An example of the procedure for selecting similar molecules 700 is provided. The protocol of the procedure selects among the set of candidates (Potential Set) the molecules that are closest (by defined metric F(x, y)) to the model set (Main Set) (see FIG. 7). The protocol can be performed by: (1) Selected Molecules—list; (2) for molecules 1 (first molecule) in Main Set: (3) Select molecules 2 (second molecule) among Potential Set with minimal value of F(mol1,mol2), the similarity metric F; (4) Delete molecule 2 from Potential Set P; and Append molecule 2 for Selected Molecules list. The Selected Molecules list is the group of selected molecules with the local latent spacing, which are similar.

In some embodiments, molecules can be characterized by descriptors-based similarity function. The molecules are characterized by a descriptor vector that reflects the chemical properties of the molecule. It is calculated by six descriptors: (1) HBA—number of hydrogen bond acceptors; (2) HBD—number of hydrogen bond donors; (3) Log P—the partition coefficient of a molecule between aqueous and lipophilic phases; (4) TopoPSA—topological polar surface area; (5) Zagreb—zagreb index of molecule (For a (molecular) graph, the first Zagreb index is equal to the sum of squares of the degrees of vertices, and the second Zagreb index is equal to the sum of the products of the degrees of pairs of adjacent vertices); and (6) SS—common electro topological index (both electronic and topological characteristics are combined). All descriptors can be calculated using the RDKit library. The use of these descriptors is justified by the fact that they can be calculated rather quickly, in contrast to the objective function. In doing so, they reflect the chemical similarity of the molecules.

The number of hydrogens boding donors and acceptors can be counted. The partition coefficient can be looked up or calculated based on the hydrophobic and hydrophilic properties of the molecule. The topological polar surface area is obtained by subtracting from the molecular surface the area of carbon atoms, halogens, and hydrogen atoms bonded to carbon atoms (i.e., nonpolar hydrogen atoms). In other words, the PSA is the surface associated with heteroatoms (namely oxygen, nitrogen, and phosphorous atoms) and polar hydrogen atoms. The Zagreb index can be calculated as known to the skilled artisan [4]. The electro topological index can be calculated as known to the skilled artisan [5].

Each of these descriptors is normalized by the formula:

$${d}_{norm} = \frac{d - {d}_{mean}}{{d}_{std}}$$

where d—value of descriptor before normalization, dₘₑₐₙ and d_{std}—mean and standard deviation of the descriptor value calculated on the training sample.

Similarity metric between molecules, based on characterization vector V=(d_{HBA}, d_{HBD}, d_{Log P}, d_{TopoPSA}, d_{MW}, d_{SS}), is calculated as follows:

$$Similarity ( {V}_{1} , {V}_{2} ) = \sqrt{{\sum }_{i = 1}^{6} {( {V}_{1 i} - {V}_{2 i} )}^{2}}$$

This is the descriptor similarity metric.

In some embodiments, descriptor-based molecule filtration can be performed. Descriptor-based molecules filtering protocol is applicable for any generative model (ABGM) with a base (e.g., database) of molecules with calculated objective function values. The descriptor-based molecule filtration protocol can include the following steps: (1) Model generates a batch of candidate molecules; (2) Calculate the descriptor vector for generated molecules; (3) Select diverse molecules using a diverse molecules selection procedure from the base of molecules with a calculated objective function; (4) Calculate the descriptor vectors for selected molecules; (5) Use a similar molecules selection procedure to obtain selected molecules as a main set of molecules, generated molecules as a potential set and descriptor similarity as F(x, y); and (6) Calculate target function only for filtered molecules. This protocol allows for any generative model to carry out an initial filtering, which will allow potentially bad molecules to be discarded based on their similarity to already known molecules with high objective function value.

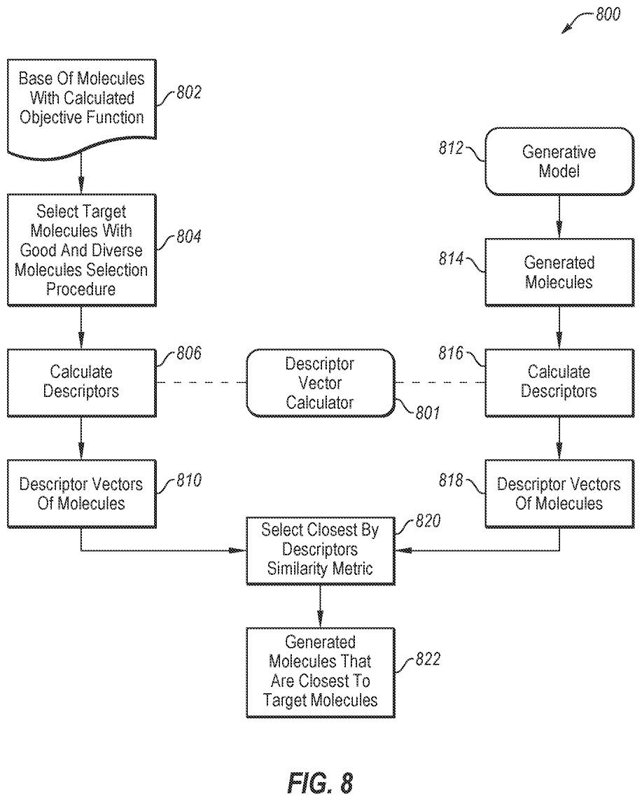

FIG. 8 illustrates an embodiment of a descriptor-based molecule filtration protocol 800. The protocol 800 can be performed as described herein. The database of molecules with the calculated objective function is provided (block 802). The target molecules are selected using the good and diverse molecules selection protocol described herein in FIG. 4 (block 804). The descriptors are calculated for the selected target molecules with the descriptor vector calculator 801 (block 806). The descriptor vectors of each of the target molecules are obtained (block 810). The generative model (ABGM) is provided (block 812) and used to generate molecules as described herein (block 814). The descriptors are calculated for the generated molecules by the description vector calculator 801 (block 816). The descriptor vectors of the generated molecules are determined (block 818). The descriptor vectors of the diverse target molecules of block 810 are compared with the descriptor vectors of the generated molecules of block 818. For example, both the target molecules and generated molecules can have the descriptor vectors analyzed for selection the closest of the target molecules and/or generated molecules by the descriptors similarity metric. The generated molecules with the closest descriptors similarity metric are selected (block 820) to obtain the generated molecules that are closest to the target molecules (block 822). Thus, the generated molecules with property of the target molecules can be obtained.

The overall computer conditional generation process methodology as described herein can be performed with computer-implemented method steps. The methods can be performed with: an autoencoder-based generative model; a high-scored and diverse molecules selection procedure (e.g., good and diverse molecules selection which can be a scored diverse selection procedure); a procedure for sampling new points in latent space; and a local steps in latent space (LSLS) molecules generation protocol. See FIGS. 1, 2 and 8.

In some embodiments, the methods can include the computing system receiving or accessing a training dataset of training molecules to train an autoencoder-based generation model (ABGM). The computing system can be configured for receiving or accessing a dataset of molecules that have a calculated objective function value that has a high score. For example, see FIG. 3. The computing system can be configured for using these molecules with the objection functions use in the high-scored good and diverse molecules selection procedure. The high-scored good and diverse molecules selection can be performed by applying a fingerprints-based clustering and sampling protocol, such as in FIG. 4. In some aspects, the sampling new points in latent space is performed by applying a latent neighbor sampling protocol, such as in FIG. 5, for the local steps in latent space protocol. In some aspects, the SMILES format or other chemical line notation can be used as a representation of molecules for the methods described herein. In some aspects, any graph representation can be used as a representation of molecules for the methods described herein.

In some embodiments, the computing system can be configured for using an adversarial autoencoder as the autoencoder-based generative model. Alternatively, a variational autoencoder can be used as the autoencoder-based generative model.

In some embodiments, the overall computer conditional generation process can be performed with a descriptors-based molecules filtration protocol (see, FIGS. 3, 7, and 8). The descriptors-based molecules filtration protocol can be implemented by: using a generative model; performing a high-scored and diverse molecules selection procedure; performing a procedure for selecting molecules from a set of candidates; computing a function for molecules similarity; and processing data with a molecules filtration protocol. In some aspects, this can include receiving a training dataset of training molecules to train autoencoder-based generation model. The descriptors-based molecules filtration protocol can include receiving a dataset of molecules with calculated objective function for a high-scored and diverse molecules selection procedure. In some aspects, the high-scored and diverse molecules selection is performed by applying fingerprints-based clustering and sampling protocol or other good and diverse molecule selection protocol. In some aspects, a process of selecting molecules from a set of candidates is performed by using a similar molecules selection protocol. In some aspects, a molecules similarity calculation is performed by using a descriptors-based similarity function. In some aspects, the SMILES format or other chemical line notation is used as a representation of molecules in the methods and protocols, such as be being used in the protocols. Alternatively, any graph representation is used as a representation of molecules. In some aspects, the methods can be performed by using an adversarial autoencoder as generative model. The methods can also be performed by using a variational autoencoder as generative model. A generative adversarial network can be used as the generative model.

In some embodiments, a method of generating molecular structures is performed on a computing system in accordance with the embodiments described herein. Such a method can include providing an autoencoder-based generative model for generation of molecular structures. Such a model can be used by inputting into the model a base of scored molecules. The method can identify molecules with relatively larger values over other molecules with an objective function. Molecules with an acceptance function value of 1 can be selected, such as when the molecules are acceptable with an acceptance function. The molecular data can be processed through an encoder to obtain latent data of molecular structures. The neighbor data points of molecular structures can be selected from the latent data in the latent space. That is, latent data points close to the latent data points of select molecular structures can be selected with an LSLS protocol. The sampled neighbor data points of the molecular structures can be processed with a decoder in order to generate at least one generated molecule.

In some embodiments, a method of generating molecular structures can be performed as follows. An autoencoder-based generative model can be provided for generation of molecular structures, such as the ABGM described herein. The model can be configured for use in the ABMG and the protocols described herein by inputting a base of scored molecules. The molecular data can be processed through an encoder in order to obtain latent data points of the molecular structures (e.g., having the high objective function value). The neighbor data points of the data point of the provided molecular structures can be selected from the latent data. That is, the latent space can include latent data points for molecules with a high objective function value, and neighboring latent data points that are close, or at least as close as being within a defined distance away, can be selected out. These selected out neighboring latent data points can be used for the processing and generating of newly generated molecules. The sampled neighbor latent data points of molecular structures can be decoded into generated structures with a decoder. Accordingly, the protocol can result in the decoder generating at least one generated molecule, which can be from the neighboring latent data points by LSLS. The molecules with relatively larger values of the objective function value can be identified and selected over other molecules with a lower objective function value. Those molecules with an acceptance function value of 1 can be identified and selected. When molecules have an acceptance function value of 1, they are acceptable with acceptance function. One or more of the generated molecules having higher objective function values can be selected and saved. One or more of the generated molecules with an acceptance function equal to 1 can be selected and provided. These generated molecules can then be validated as having the property. The method can be performed with an object function of:

OF: x→R.

The larger the value of this objective function, the more suitable the molecule is for having the desired one or more properties.

Also, the method can be performed with an acceptance function of:

F: x→{0,1};

Here, if AF(x)=1, then the x molecule is acceptable, otherwise AF(x)=0 and the molecule is not acceptable. Thus, the acceptance function can be calculated for use in filtering out molecules that do not fit the criteria, and not equal to 1.

In some embodiments, the methods can be performed with one of more of the following steps. Acceptable molecules with AF(x)=1 are selected. A chemical fingerprint for the selected molecules can be determined. The clustering method can be applied on the calculated fingerprints. In every cluster, there are N molecules that are selected that each have a high or highest value of the objective function. This can help generate molecules with the one or more desired properties. The protocol can include randomly selecting certain molecules from the selected molecules, which can be to randomly choose one molecule in every cluster. In some aspects, the fingerprint is a Morgan fingerprint, extended connectivity fingerprint (ECFP), or other fingerprint. In some aspects, the selected molecules are a subset of molecules with higher objective function values compared to other molecules that are not selected. Low objective function value molecules can be omitted.

In some embodiments, a latent neighbor sampling protocol can be performed. This can be in the latent space for obtaining candidates for neighboring latent data points. In some aspects, the latent neighbor sampling protocol includes: determine distance=L; obtain the Sampled Points which is initially an empty list; For k in 1; N do: Sample S random points in Latent Space—Neighbors, for every Point from Neighbors d(Point, Start)=Distance, where d is an Euclidean distance; add points to Sampled Points (e.g., no longer an empty list; and select a new point with distance=distance+L. The latent neighbor sampling protocol can be used with a point from Latent Space (Start), step length (L), number of levels (N); L is the distance of the neighboring latent data point to the first latent data point; and number of steps per level (S). The k can be for each property.

In some embodiments, methods are provided for identifying candidates for drugs that have a defined biological activity. The methods can be performed as described herein. A generative model can be provided and processed to generate generated molecules. A database of scored molecules can be provided or accessed by the generative model, which score is the objective function value. A selection of molecules protocol can be performed in order to obtain different molecules with high scores for the objective function. Then, from the generated molecules and the selected molecules, the protocol can select the generated molecules that are the closest to a high score of the selected molecules. These generated molecules can have the desired property. The selected generated molecules can be identified as candidates for a drug. Thus, these generated molecules can be obtained in physical copies and assayed for function as the drug. Also, these generated molecules can be simulated in a digital simulator of a biological functionality to determine if there is any modulation of the biological functionality to indicate the generated molecule can function as the desired drug.

In some embodiments, methods can be provided for the identification of newly generated molecules that have a defined property, such as any property described herein. In some aspects, the generated molecules that have one or more defined properties can be identified and selected. The methods can be performed as follows. A base (e.g., a database with data of molecules having at least one defined property with an objective function value) of molecules, each with a calculated objective function value can be used. Or, the method can include calculating the objective function and introducing these molecules with the objective function value into the base of molecules, which can update the base. Molecules with an acceptance function value of 1 can be selected, and the rest can be discarded or placed into an excluded bin. The chemical fingerprints can be calculated for each selected molecule. A clustering function can be performed to cluster the selected molecules by fingerprint vector. As a result, top molecules can be selected from each cluster, such as having the highest objective function. As such, the protocol can include sorting the selected molecules by objective function value. For example, the protocol can result in selecting the molecules with a relatively higher objective function value in each cluster or randomly sample one molecule in each cluster. This can provide for the generated molecule that is selected to have the defined property.

In some embodiments, a method of one of the embodiments described herein can be performed as follows: determine L in step length; determine N number of levels; determine S number of steps for each level; define distance equal to L; define sampled points to initially be equal to an empty list; i starts at 0, when i less than N, perform the following: sample S random points in latent space; identify neighbors for the S random points in latent space; then, for every point from identified neighbors, determine distance, such as Euclidian distance; add sampled points to a Sampled Points, which is a database for the sampled points; in the next iteration, then i is i+1; and then the distance is distance+L; when i is greater than N, then obtain the Sampled Points database. The Sampled Points database includes newly generated molecules that have the defined property.

In some embodiments, a method of generating molecules with a property can be performed with the protocol as recited: providing an autoencoder based generative model (ABGM); training ABGM model; selecting molecules using selection protocol; encoding molecules to latent points using ABGM encoder; selecting neighbor latent points in latent space using a local step in latent space protocol; decoding neighbor latent points to obtained generated molecules using an ABGM decoder; filtering new and valid molecules, such as filtering for molecules that have the property; selecting generated molecules that are closest in latent space to the target molecules; calculating an objective function for the generated molecules; optionally adding generated molecules to database; and providing the generated molecules with the highest calculated objective function (e.g., above a threshold or a highest percentage). In some aspects, generated molecules in the molecules database with calculated objective functions and/or the generated molecules with objective functions are used for training the ABGM model.

In some embodiments, the methods described herein can include performing a protocol as follows: Train an AGBM on data from the base of molecules; Select diverse molecules using a diverse molecules selection procedure from the base of molecules with a calculated objective function—target molecules; Encode target molecules to AGBM latent space using the encoder; Select neighbor latent points in latent space using the local steps in latent space protocol (FIG. 2); Decode neighbor latent points to molecules using the decoder; Filter for only new and valid generated molecules; Select the molecules that are the closest to target molecules by Euclidean distance in latent space; Calculate the objective function for selected generated molecules and add generated molecules to the base; Return to the training step or provide the selected generated molecules in a report. The report can identify the selected generated molecules that have a defined property.

In some embodiments, the molecules are characterized by a descriptor vector that reflects the one or more desired chemical properties of the molecule. In some aspects, the method can include calculating one or more of the following six descriptors: HBA—hydrogen bond acceptors; HBD—hydrogen bond donors; Log P—the partition coefficient of a molecule between aqueous and lipophilic phases; TopoPSA—topological polar surface area; Zagreb—zagreb index of molecule; and SS—common electro topological index.

In some embodiments, the methods described herein can include: providing a model that generates a batch of candidate molecules; calculating the descriptor vector for generated molecules; selecting diverse molecules using the a diverse molecules selection procedure with a high score from the base of molecules with a calculated objective function values; calculating the descriptor vectors for selected molecules; and selecting molecules using a similar molecules selection procedure to obtain selected molecules as a main set of generated molecules. The generated molecules can be labeled as a potential set and studied for the descriptor similarity function as F(x, y). The target function can be calculated only for filtered molecules.

The methodologies provided herein can be performed on a computer or in any computing system. In some embodiments, the computer can include generative adversarial networks that are adapted for conditional generation of objects (e.g., generated objects), when a known external variable, such as the condition/property, influences and improves generation and decoding. When data consists of pairs of complex objects, e.g., a supervised dataset with a complex condition/property for a molecule, the computing system can create a generated complex object (e.g., molecules) that is similar to the provided complex object (e.g., provided molecule) of the data that satisfies the complex condition/property (e.g., biological activity, physiochemical property, etc.) of the data. The computing system can process the models described herein that are based on the adversarial autoencoder architecture that can learn three latent representations: (1) object/molecule only information; (2) condition/property only information, and (3) common information between the object/molecule and the condition/property. The model can be validated or trained with a dataset of molecules with a high objective function for the property, where common information is a digit, and then apply the training to a practical problem of generating fingerprints of molecules with desired properties. In addition, the model is capable of metric learning between objects and conditions without negative sampling.

The condition usually represents a target variable, such as a class label in a classification problem, which represents one or more desired properties. In an example, the condition “y” is a complex object itself, such as biological activity. For example, drug discovery is used to identify or generate specific molecules with a desired action on human cells (e.g., such a property), or molecules that bind to some protein. In both cases, the condition (e.g., protein binding) is at least as complex as the object (e.g., a candidate molecule for a drug) itself. The protocols described herein can be applied to any dataset of object/property pairs (x, y). When a computing process operates with the models described herein, the computer can extract common information from the object and the condition/property and rank generated objects by their relevance to a given condition and/or rank generated conditions by their relevance to a given object.

The model includes the encoders performing a decomposition of the object data and condition data to obtain the latent representation data. The latent representation data is suitable for conditional generation of generated objects and generated conditions/properties by the generators and may also be suitable for use in metric learning between objects and conditions.

As used herein, the model includes encoders Eₓ and E_{y}, a generators Gₓ and G_{y} (i.e., decoders), and “x” is the object molecule, “y” is the condition/property, and all z correspond to the latent representations produced by the encoders. The model can be applied to a problem of mutual conditional generation of “x” and “y” given a dataset of pairs (x, y). Both x and y can be assumed to be complex, each containing information irrelevant for conditional generation of the other.

### EXAMPLES

The protocols require two main molecular data collections.

The generative model pretrain database was used. This database is a collection of molecules in a representation that the generative model takes as input. The data can be SMILES or any line notation scheme, or another mathematical representation of a molecule. These molecules do not require a calculated value of objective function, because the generative model is only pre-trained to generate molecules. But it is desirable for the distribution of these molecules to be similar to the distribution of the molecules to be generated, such as the molecules with high value of objective function.

Also, the database of molecules with calculated objective function was used. The data for molecules with the calculated objective function is needed directly for the molecule generation process and for the protocol of training the model. The training can be prior to or during a molecule generating or selecting protocol. Molecules from this collection must have an objective function that has calculated. The generated molecules that have a calculated objective function obtained therefor during training or molecule generation/selection protocols can be added to the collection in order to update the collection. In the local steps in latent space molecular generation protocol, these molecules with the objective function values are used to encode and create latent points. For the descriptors-based molecules filtering protocols, molecules from this collection are used to create a set of target molecules. Also, this part of the data is used to train the model before and during the molecule generation or selection process.

### EXPERIMENTS

The GuacaMol benchmark [3] was used to test the protocols. This benchmark allows one to evaluate molecular generative models by various parameters, including goal-oriented generation. GuacaMol is an open-source Python package for benchmarking of models for de novo molecular design, which is incorporated herein by specific reference.

Implementation of the protocols was based on the following components: (1) SMILES format as input representation of molecules; (2) Adversarial Autoencoder (AAE) based on LSTM layers as a generative model; (3) GuacaMol train dataset as data for pre-training generative model; and (4) Scored molecules from GuacaMol train dataset as initial state of base of molecules with calculated objective function.

There are 7 goal-oriented tasks from the benchmark. Each of the tasks has some target drug molecule (e.g., Osimertinib, Fexofenadine, Ranolazine, Perindopril, Amlodipine, Sitagliptin, Zaleplon). Generative models should generate molecules that are similar to it. The objective function is the similarity of the molecule with the target: OF(x)=Similarity(x, target), OF: x→[0,1]. The final quality of the models is calculated according to the following formula: Metric=⅓(s₁+ 1/10Σᵢ₌₁¹⁰ sᵢ+ 1/100Σᵢ₌₁¹⁰⁰ sᵢ), where s is a 100-dimensional vector of molecule scores sᵢ, 1≤i≤100, sorted in decreasing order (i.e., sᵢ≥sⱼ for i<j). More details are provided in the original benchmark article (3).

The protocols were compared with the models that are presented in the basic version of the benchmark. Since the protocols were applied to the LSTM-based autoencoder with SMILES representation of molecules, a comparison was made with the models corresponding to this approach: SMILES LSTM and SMILES GA.

The results of the protocols and their comparison with the benchmark models are presented in Table 1 (LSLS AAE—AAE with Local Steps in Latent Space generation protocol). The (LSLS AAE protocol demonstrated results better than SMILES GA model and comparable to the results of SMILES LSTM—on 3 out of 7 tasks, LSLS AAE got the best quality, on the remaining 4, SMILES LSTM worked better. This shows the improvement to the technology with the present invention.

TABLE 1

| Target | SMILES LSTM | SMILES GA | LSLS AAE |
|---|---|---|---|
| Osimertinib MPO | 0.907 | 0.886 | 0.908 |
| Fexofenadine MPO | 0.959 | 0.931 | 0.939 |
| Ranolazine MPO | 0.855 | 0.881 | 0.906 |
| Perindopril MPO | 0.808 | 0.661 | 0.748 |
| Amlodipine MPO | 0.894 | 0.722 | 0.849 |
| Sitagliptin MPO | 0.545 | 0.689 | 0.833 |
| Zaleplon MPO | 0.669 | 0.413 | 0.629 |

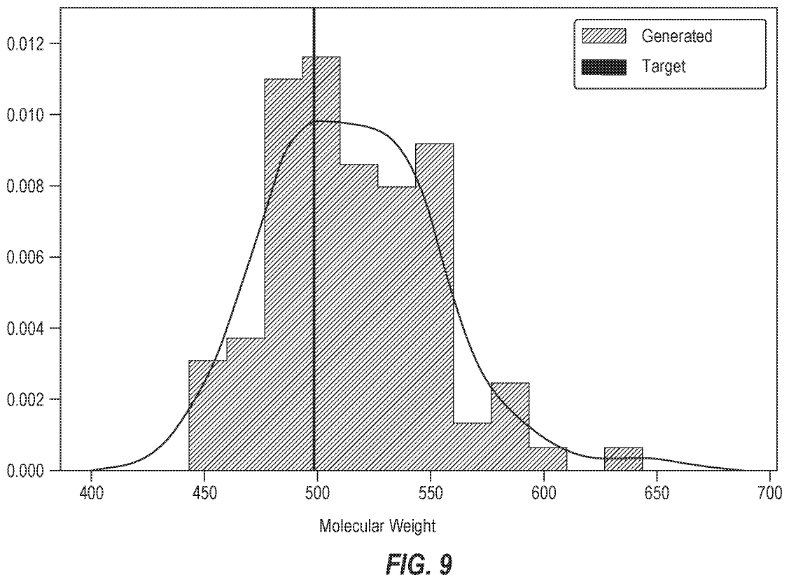

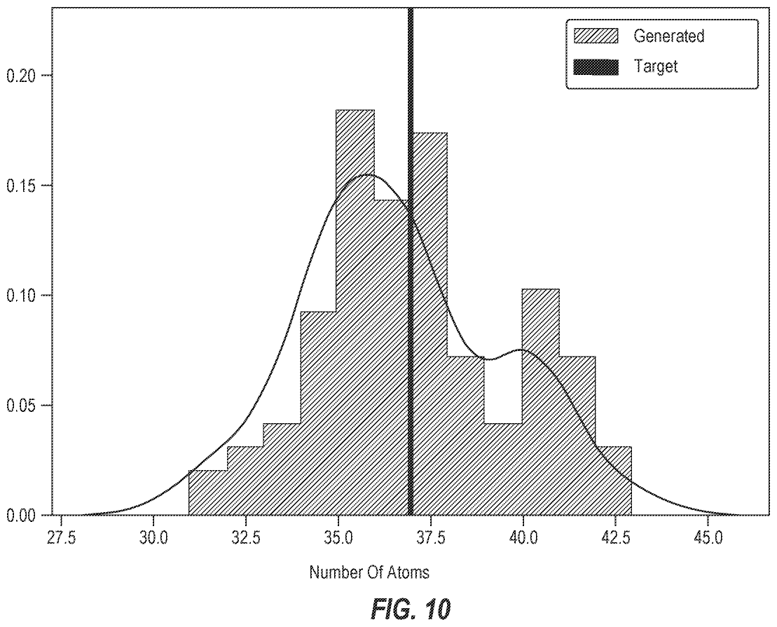

FIG. 9 compares the distribution of the number of atoms in the molecules generated by LSLS AAE for the Osimertinib MPO task with the Osimertinib number of atoms. FIG. 10 compares the molecular weight distribution of the molecules generated by the LSLS AAE for the same task with the molecular weight of Osimertinib.

One skilled in the art will appreciate that, for the processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In one embodiment, the present methods can include aspects performed on a computing system. As such, the computing system can include a memory device that has the computer-executable instructions for performing the methods. The computer-executable instructions can be part of a computer program product that includes one or more protocols or algorithms for performing any of the methods of any of the claims.

In one embodiment, any of the operations, processes, or methods, described herein can be performed or cause to be performed in response to execution of computer-readable instructions stored on a computer-readable medium and executable by one or more processors. The computer-readable instructions can be executed by a processor of a wide range of computing systems from desktop computing systems, portable computing systems, tablet computing systems, hand-held computing systems, as well as network elements, and/or any other computing device. The computer readable medium is not transitory. The computer readable medium is a physical medium having the computer-readable instructions stored therein so as to be physically readable from the physical medium by the computer/processor.

There are various vehicles by which processes and/or systems and/or other technologies described herein can be effected (e.g., hardware, software, and/or firmware), and that the preferred vehicle may vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The various operations described herein can be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and/or firmware are possible in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a physical signal bearing medium include, but are not limited to, the following: a recordable type medium such as a floppy disk, a hard disk drive (HDD), a compact disc (CD), a digital versatile disc (DVD), a digital tape, a computer memory, or any other physical medium that is not transitory or a transmission. Examples of physical media having computer-readable instructions omit transitory or transmission type media such as a digital and/or an analog communication medium (e.g., a fiber optic cable, a waveguide, a wired communication link, a wireless communication link, etc.).

It is common to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. A typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems, including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. Such depicted architectures are merely exemplary, and that in fact, many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include, but are not limited to: physically mateable and/or physically interacting components and/or wirelessly interactable and/or wirelessly interacting components and/or logically interacting and/or logically interactable components.

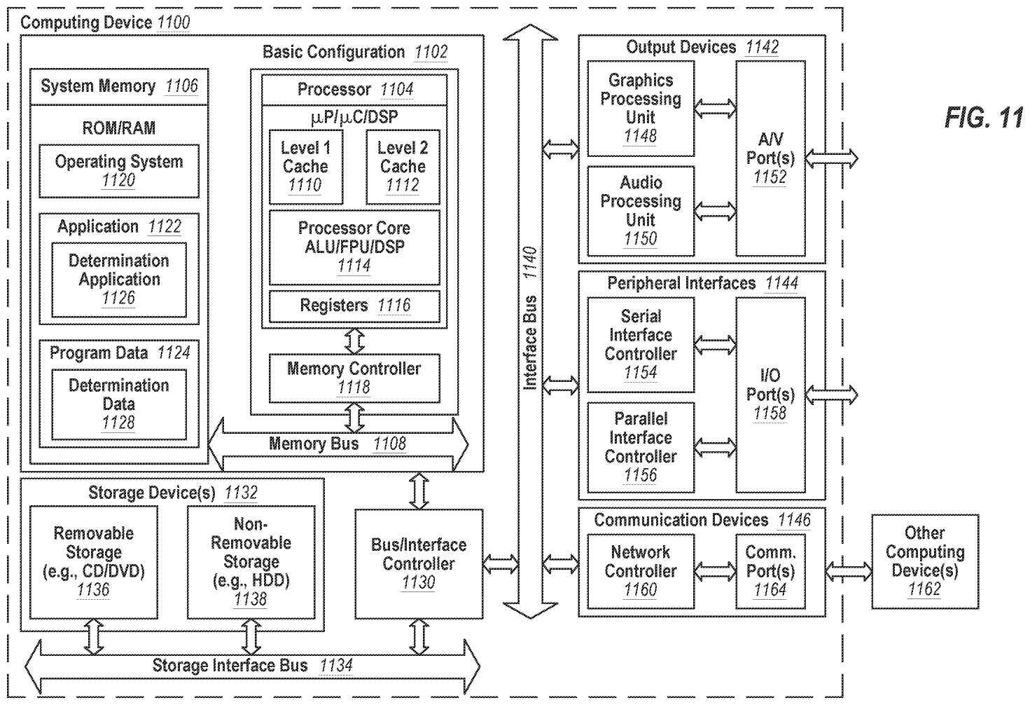

FIG. 11 shows an example computing device 1100 (e.g., a computer, a system thereof, cloud computing system, or any computing system known or developed) that may be arranged in some embodiments to perform the methods (or portions thereof) described herein. In a very basic configuration 1102, computing device 1100 generally includes one or more processors 1104 and a system memory 1106. A memory bus 1108 may be used for communicating between processor 1104 and system memory 1106.

Depending on the desired configuration, processor 1104 may be of any type including, but not limited to: a microprocessor (μP), a microcontroller (μC), a digital signal processor (DSP), or any combination thereof. Processor 1104 may include one or more levels of caching, such as a level one cache 1110 and a level two cache 1112, a processor core 1114, and registers 1116. An example processor core 1114 may include an arithmetic logic unit (ALU), a floating-point unit (FPU), a digital signal processing core (DSP Core), or any combination thereof. An example memory controller 1118 may also be used with processor 1104, or in some implementations, memory controller 1118 may be an internal part of processor 1104.

Depending on the desired configuration, system memory 1106 may be of any type including, but not limited to: volatile memory (such as RAM), non-volatile memory (such as ROM, flash memory, etc.), or any combination thereof. System memory 1106 may include an operating system 1120, one or more applications 1122, and program data 1124. Application 1122 may include a determination application 1126 that is arranged to perform the operations as described herein, including those described with respect to methods described herein. The determination application 1126 can obtain data, such as pressure, flow rate, and/or temperature, and then determine a change to the system to change the pressure, flow rate, and/or temperature.

Computing device 1100 may have additional features or functionality, and additional interfaces to facilitate communications between basic configuration 1102 and any required devices and interfaces. For example, a bus/interface controller 1130 may be used to facilitate communications between basic configuration 1102 and one or more data storage devices 1132 via a storage interface bus 1134. Data storage devices 1132 may be removable storage devices 1136, non-removable storage devices 1138, or a combination thereof. Examples of removable storage and non-removable storage devices include: magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include: volatile and non-volatile, removable and non-removable media implemented in any method or technology for storage of information, such as computer readable instructions, data structures, program modules, or other data.

System memory 1106, removable storage devices 1136 and non-removable storage devices 1138 are examples of computer storage media. Computer storage media includes, but is not limited to: RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 1100. Any such computer storage media may be part of computing device 1100.

Computing device 1100 may also include an interface bus 1140 for facilitating communication from various interface devices (e.g., output devices 1142, peripheral interfaces 1144, and communication devices 1146) to basic configuration 1102 via bus/interface controller 1130. Example output devices 1142 include a graphics processing unit 1148 and an audio processing unit 1150, which may be configured to communicate to various external devices such as a display or speakers via one or more A/V ports 1152. Example peripheral interfaces 1144 include a serial interface controller 1154 or a parallel interface controller 1156, which may be configured to communicate with external devices such as input devices (e.g., keyboard, mouse, pen, voice input device, touch input device, etc.) or other peripheral devices (e.g., printer, scanner, etc.) via one or more I/O ports 1158. An example communication device 1146 includes a network controller 1160, which may be arranged to facilitate communications with one or more other computing devices 1162 over a network communication link via one or more communication ports 1164.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR), and other wireless media. The term computer readable media as used herein may include both storage media and communication media.

Computing device 1100 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 1100 may also be implemented as a personal computer including both laptop computer and non-laptop computer configurations or as a cloud computing system or any other computing system. The computing device 1100 can also be any type of network computing device. The computing device 1100 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In some embodiments, a computer program product can include a non-transient, tangible memory device having computer-executable instructions that when executed by a processor, cause performance of a method that can include at least one of: providing a dataset having object data for an object and property data for a property; processing the object data of the dataset to obtain latent object data and latent object-property data with an object encoder; processing the property data of the dataset to obtain latent property data and latent property-object data with a property encoder; processing the latent object data and the latent object-property data to obtain generated object data with an object decoder; processing the latent property data and latent property-object data to obtain generated property data with a property decoder; comparing the latent object-property data to the latent-property data to determine a difference; processing the latent object data and latent property data and one of the latent object-property data or latent property-object data with a discriminator to obtain a discriminator value; selecting a selected object from the generated object data based on the generated object data, generated property data, and the difference between the latent object-property data and latent property-object data; and providing the selected object in a report with a recommendation for validation of a physical form of the object. The non-transient, tangible memory device may also have other executable instructions for any of the methods or method steps described herein. Also, the instructions may be instructions to perform a non-computing task, such as synthesis of a molecule and or an experimental protocol for validating the molecule. Other executable instructions may also be provided.

An autoencoder (AE) is a type of deep neural network (DNN) used in unsupervised learning for efficient information coding. The purpose of an AE is to learn a representation (e.g., encoding) of objects (e.g., molecules). An AE contains an encoder part, which is a DNN that transforms the input information from the input layer to the latent representation (e.g., latent code, latent data point), and includes a decoder part, which uses the latent representation and decodes an original object with the output layer having the same dimensionality as the input object for the encoder. Often, a use of an AE is for learning a representation or encoding for a set of data. An AE learns to compress data from the input layer into a short code, and then un-compress that code into something that closely matches the original data. In one example, the original data may be a molecule that interacts with a target protein (e.g., property), and thereby the AE can design a molecule that is not part of an original set of molecules or select a molecule from the original set of molecules or variation or derivative thereof that interacts (e.g., binds with a binding site) of the target protein.

Generative Adversarial Networks (GANs) are structured probabilistic models that can be used to generate data. GANs can be used to generate data (e.g., a molecule) similar to the dataset (e.g., molecular library) GANs are trained on. A GAN can include two separate modules, which are DNN architectures called: (1) discriminator and (2) generator. The discriminator estimates the probability that a generated product comes from the real dataset, by working to compare a generated product to an original example, and is optimized to distinguish a generated product from the original example. The generator outputs generated products based on the original examples. The generator is trained to generate products that are as real as possible compared to an original example. The generator tries to improve its output in the form of a generated product until the discriminator is unable to distinguish the generated product from the real original example. In one example, an original example can be a molecule of a molecular library of molecules that bind with a protein (e.g., property), and the generated product is a molecule that also can bind with the protein (e.g., thereby having the property), whether the generated product is a variation of a molecule in the molecular library or a combination of molecules thereof or derivatives thereof.

Adversarial Autoencoders (AAEs) are probabilistic AEs that use GANs to perform variational inference. AAEs are DNN-based architectures in which latent representations are forced to follow some prior distribution via the discriminator.

A conditional architecture may be considered a supervised architecture because the processing is supervised by the condition (e.g., a molecule having the property). As such, the conditional architecture may be configured for generating objects that match a specific condition (e.g., property of molecule). In some applications, a conditional model can take values of conditions into account, even if the values of conditions are only partially known. During the generation process, the conditional architecture may only have a few conditions that are specified, and thereby the rest of the conditions can take arbitrary values, at least initially.

A subset conditioning problem is defined as a problem of learning a generative model with partially observed conditions during training and/or generation (active use). The architecture described herein, which can be used for a subset conditioning problem, is a variational autoencoder-based generative model extended for conditional generation.

Generally, the present technology relates to generative models that are configured to produce realistic objects (e.g., chemicals, phrases, pictures, audio, video, etc.) in many domains including chemistry, text, images, video, and audio. However, some applications, for example in the field of chemistry, such as for biomedical applications where the missing data (e.g., property of molecule) is a common issue, require a model that is trained to condition on multiple properties with some of the properties being unknown during the training or generation procedure. Accordingly, references to generation and selection of molecules can be applied to these other objects, and thereby the present methods also relate to these other objects.

The autoencoder can be configured to generate objects with a specific set of properties, where the object can be an image, video, audio, molecules, or other complex objects. The properties of the objects themselves may be complex and some properties may be unknown. The autoencoder can be considered to be a model that undergoes two phases, which are (1) training the model with objects with object-specific properties, and then using the trained model to (2) generate objects that are indistinguishable from the objects used to train the model and which also satisfy the properties. Also, during the generation process using the model, the operator of the model can specify only a few properties, allowing the rest of properties to take arbitrary values. For example, the autoencoder can be particularly useful for reconstructing lost or deteriorated parts of objects, such as lost parts of images, text, or audio. In such cases, a model can be trained to generate full objects (e.g., images) conditioned on observed elements. During the training procedure, the model is provided access to full images, but for the generation, the operator may specify only observed pixels as a condition (e.g., property). The similar problem appears in drug discovery, where the operator uses the model to generate new molecular structures with predefined properties, such as activity against a specific target or a particular solubility. In most cases, the intersection between measured parameters in different studies is small, so the combined data from these studies have a lot of missing values. During the generation, the operator might want to specify only the activity of a molecule as a property, so the resulted solubility of generated molecules can initially take an arbitrary value. Here, the process will have missing values in properties during training as well as in generation procedures.

In some embodiments, a method is provided for generating new objects having given properties. That is, the generated objects have desired properties, such as a specific bioactivity (e.g., binding with a specific protein). The objects can be generated as described herein. In some aspects, the method can include: (a) receiving objects (e.g., physical structures) and their properties (e.g., chemical properties, bioactivity properties, etc.) from a dataset; (b) providing the objects and their properties to a machine learning platform, wherein the machine learning platform outputs a trained model; and (c) the machine learning platform takes the trained model and a set of properties and outputs new objects with desired properties. The new objects are different from the received objects. In some aspects, the objects are molecular structures, such as potential active agents, such as small molecule drugs, biological agents, nucleic acids, proteins, antibodies, or other active agents with a desired or defined bioactivity (e.g., binding a specific protein, preferentially over other proteins). In some aspects, the molecular structures are represented as graphs, SMILES strings, fingerprints, InChI or other representations of the molecular structures. In some aspects, the object properties are biochemical properties of molecular structures. In some aspects, the object properties are structural properties of molecular structures.

In some embodiments of the method for generating new objects having given properties, the machine learning platform consists of two or more machine learning models. In some aspects, the two or more machine learning models are neural networks, such as fully connected neural networks, convolutional neural networks, or recurrent neural networks. In some aspects, the machine learning platform includes a trained model that converts a first object into a latent representation, and then reconstructs a second object (e.g., second object is different from the first object) back from the latent codes. In some aspects, the machine learning platform enforces a certain distribution of latent codes across all potential objects. In some aspects, the model uses adversarial training or variational inference for training. In some aspects, the model that uses a separate machine learning model to predict object properties from latent codes.

In some embodiments, the object can be any type of object in view of the examples of image, video, audio, text, and molecule. As such, the object can be anything that is represented by data which can be perceived by human. Accordingly, the object data can include the data that defines that which is perceived by the human. Further examples of the object can include biological data, such as biological data profiles of genomics, transcriptomics, proteomics, metabolomics, lipidomics, glycomics, or secretomics, as well as combinations thereof or others. Any omic biological data signature may be an object. For example, a gene expression profile can be a genomic biological data signature. A protein signature can also be an object that shows the proteomic profile, which can be obtained from a biological sample.

In some embodiments, the object is a molecule, such as a small molecule, macromolecule, polypeptide, protein, antibody, oligonucleotide, nucleic acid (e.g., RNA, DNA, etc.), polypeptide, carbohydrate, lipid, or combinations thereof, whether natural or synthetic.

In some embodiments, the image, video, audio, or text objects can have suitable properties related thereto, such as the content thereof. These types of objects can have properties consistent with the type of information usually present. Images can include scenery that includes common environmental features, whether natural (e.g., sky, earth, plants, animals, etc., or man-made such as buildings, roads, articles of manufacture, and ornamentals. Video can include the properties of images in a sequence of images with or without sounds corresponding to the imagery in the video. Audio can include sounds of any type, from animal sounds, such as human voice, as well as music, and natural environment sounds (e.g., river, ocean, wind, thunder, etc.). Text can include properties of words, phrases, sentences, paragraphs, chapters, and any type of textual language subject matter.

In some embodiments, the property can be biological activity of the object, such as the biological response to the property, which may be a modulation of any of transcriptomic data profile, proteomic data profile, metabolomic data profile, lipidomic data profile, glycomic data profile, or secretomic data profile, as well as combinations thereof or others. Gene expression profiles in response to activity of an object may be an exemplary property. Also, absorption, distribution, metabolism, and excretion (ADME) or any pharmacokinetic data may be properties of an object in an organism, organ, fluid, extracellular matrix, or cell thereof. Toxicity is another example of a biological property. Any modulation of a biological pathway may be considered to be a property of an object. Additionally, the property can be physicochemical properties of the molecule types described herein. The physicochemical properties may also be molecular weight, melting point, boiling point, vapor point, molecular polarity, Henry's phase distribution, and the extrinsic properties of pressure (P) and moles (n), as well as others.

In some aspects, the object may be defined as a property as described herein, and thereby the corresponding property is the object that has that property. This shows the traditional objects and properties can be switched, such that the property is used as an object, and the object is used as a property.

In some embodiments, an object property is an activity against given target proteins. The generated object has this property of activity against one or more given target proteins. Often, the generated object specifically targets a specific target protein over other proteins (e.g., even over related proteins). In some aspects, the object property is a binding affinity towards a given site of a protein, where the generated object can have this object property. In some aspects, the object property is a molecular fingerprint, and the generated object has this object property. In some aspects, the object properties are biochemical properties of molecular structures, where the object property is a lipophilicity.

In some embodiments, the real objects are molecules, and the properties of the molecules are biochemical properties and/or structural properties. In some embodiments, the sequence data includes SMILES, scaffold-oriented universal line system (SOULS), InChI, SYBYL line notation (SLN), SMILES arbitrary target specification (SMARTS), Wiswesser line notation (WLN), ROSDAL, or combinations thereof.

In some aspects, the property is synthetic accessibility. The synthetic accessibility for the property of the molecule can be a retrosynthesis-related synthetic accessibility (ReRSA) estimation.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

Cross-reference is made to the following incorporated references: U.S. Pat. No. 11,403,521; US 2020/0090049; US 2020/0082916; US 2022/0310196; US 2021/0233621; US 2021/0271980; US 2021/0287067; US 2021/0383898; US 2022/0172802; US 2022/0406404; WO 2021/165887; and WO 2021/229454.

All references recited herein are incorporated herein by specific reference in their entirety.

### REFERENCES

1. SMILES: pubs.acs.org/doi/10.1021/ci00057a005; SMILES, a chemical language and information system. 1. Introduction to methodology and encoding rules; David Weininger; Journal of Chemical Information and Computer Sciences 1988 28 (1), 31-36; DOI: 10.1021/ci00057a005.
2. Morgan fingerprints: doi.org/10.1021/c160017a018; The Generation of a Unique Machine Description for Chemical Structures-A Technique Developed at Chemical Abstracts Service; H. L. Morgan; Journal of Chemical Documentation 1965 5 (2), 107-113; DOI: 10.1021/c160017a018.
3. GuacaMol: doi.org/10.102/acs.jcim.8b00839; GuacaMol: Benchmarking Models for de Novo Molecular Design; Nathan Brown, Marco Fiscato, Marwin H. S. Segler, and Alain C. Vaucher; Journal of Chemical Information and Modeling 2019 59 (3), 1096-1108 DOI: 10.1021/acs.jcim.8b00839.
4. Some formulae for the Zagreb indices of graphs; AIP Conference Proceedings 1479, 365 (2012); doi.org/10.1063/1.4756139; Ismail Naci Cangul, Aysun Yurttas, and Muge Togan.
5. Kier, L. B., Hall, L. H. An Electrotopological-State Index for Atoms in Molecules. Pharm Res 7, 801-807 (1990). doi.org/10.1023/A:1015952613760.

1. A method of generating molecular structures, comprising:
providing an autoencoder-based generative model for generation of molecular structures;
inputting into the autoencoder-based generative model a database of scored molecules, each scored molecule having an objective function value calculated from an objective function;
selecting scored molecules from the database with relatively larger objective function values over other scored molecules in the database;
processing the selected scored molecules through an encoder of the autoencoder-based generative model to obtain latent points in a latent space;
selecting a latent point in the latent space;
sampling neighbor latent points that are within a distance from the selected latent point;
processing the sampled neighbor latent points with a decoder to generate at least one generated molecule; and
providing a report having the at least one generated molecule.

2. The method of claim 1, wherein the scored molecules have at least one property, the method further comprising:
comparing the generated molecules with selected scored molecules;
selecting molecules from the generated molecules that are closest to the selected scored molecules; and
providing the selected molecules as candidates for having the at least one property.

3. The method of claim 2, wherein the selecting is based on at least one of:
a fingerprint molecule clustering and sampling protocol; or
an acceptance function having an acceptance function value equal to 1.

4. The method of claim 3, wherein the fingerprint molecule clustering and sampling protocol includes:
selecting scored molecules from the database that have the acceptance function value equal to 1;
calculating fingerprints for the selected scored molecules;
clustering the selected scored molecules by a fingerprint vector;
selecting a top number of molecules in each cluster;
sorting the selected top number of molecules by objective function value;
randomly sampling one molecules from each cluster; and
providing the randomly sampled molecule from each cluster in the report.

5. The method of claim 1, wherein a local steps in latent space protocol includes:
determining a latent point as a starting point;
determining a step length;
determining a number of levels;
determining a number of steps in each level;
when a number of latent points in a sampled points list is less than a threshold, perform the following:
(a) sample a number of random points in the latent space;
(b) sample neighboring points within a defined distance from the sampled random points;
(c) add the sampled neighboring points to the sample points list;
(d) increase the defined distance; and
repeat steps (a)-(d) until the number of latent points in the sampled points list is equal to the threshold, and then provide the sample points list having the threshold number of latent points.

6. The method of claim 1, comprising:
training the autoencoder-based generative model with the scored molecules;
selecting scored molecules with high objective function value that are diverse to obtain encodable molecules;
encoding the encodable molecules to latent points in the latent space using the encoder;
obtaining new latent points in the latent space that are neighboring latent points to selected latent points;
decoding the new latent points into newly generated molecules using the decoder;
calculating an objective function value for the newly generated molecules; and
updating the database of molecules with calculated objective function value with the newly generated molecules.

7. The method of claim 6, further comprising:
filtering the newly generated molecules for valid molecules; and
selecting newly generated molecules that are closest in latent space to each other.

8. The method of claim 7, wherein the newly generated molecules are selected by:
determining a property for a target molecule;
obtaining a potential set of molecules;
determining a similarity metric for the molecules in the potential set; and
selecting molecules in potential set with a similarity metric that is closest to the target molecule having the property.

9. The method of claim 1, comprising:
calculating molecular descriptors of the generated molecules;
calculating molecular descriptors of the selected molecules;
comparing molecular descriptors of the generated molecules to molecular descriptors of the selected molecules;
selecting generated molecules with molecular descriptors closest to target molecules; and
providing the selected generated molecules that are closes to target molecules.

10. The method of claim 9, further comprising:
selecting the target molecules by protocol that selects diverse molecules, wherein the protocol that selects diverse molecules comprises:
selecting scored molecules from the database that have an acceptance function value equal to 1;
calculating fingerprints for the selected scored molecules;
clustering the selected scored molecules by a fingerprint vector;
selecting a top number of molecules in each cluster;
sorting the selected top number of molecules by objective function value; and
randomly sampling one molecules from each cluster; and
providing the randomly sampled molecule from each cluster in the report.

11. The method of claim 9, further comprising:
calculating molecular descriptors as one or more of the following:
number of hydrogen bond acceptors;
number of hydrogen bond donors;
partition coefficient of a molecule between aqueous and lipophilic phases;
a topological polar surface area;
a zagreb index of molecule; or
an electro topological index.

12. The method of claim 11, further comprising:
calculating similarity metric between molecules based on the molecular descriptors; and
selecting generated molecules closest to similarity metric.

13. The method of claim 1, comprising:
selecting acceptable molecules with AF(x)=1;
calculating a chemical fingerprint for selected molecules;
applying a clustering method on the calculated fingerprints;
selecting in every cluster N molecules with highest values of objective function; and
from the selected molecules, randomly choosing one molecule in every cluster.

14. The method of claim 1, comprising:
selecting molecules with an acceptance function of 1;
calculating chemical fingerprints for each selected molecule;
clustering molecules by fingerprint vector;
selecting top molecules in each cluster;
sorting molecules by objective function; and
selecting molecules with relatively higher objective function in each cluster or randomly sample one molecule in each cluster.

15. The method of claim 1, comprising:
generating generated molecules with the generative model;
providing a base of scored molecules;
performing a selection of molecules to obtain different molecules with high scores;
from the generated molecules and the selected molecules, selecting generated molecules closest to a high score of the selected molecules; and
identifying the selected generated molecules as candidates to have at least one defined property.

16. The method of claim 1, comprising:
training the autoencoder-based generative model with the selected molecules from the database;
selecting molecules using a selection protocol;
encoding molecules to latent points using encoder;
creating new points in latent space using a latent space making step protocol;
decoding the new latent points to molecules using decoder;
filtering new and valid molecules;
selecting molecules that are closest in latent space molecules;
calculating objective function; and
adding generated molecules to the database.

17. The method of claim 1, comprising:
obtaining a batch of candidate molecules from the at least one generated molecule;
calculating a descriptor vector for each candidate molecule;
selecting diverse molecules from a cluster of molecules sorted by objective function value;
calculating the descriptor vectors for selected diverse molecules;
calculating similarity metric between molecules based on the molecular descriptors; and
selecting generated molecules closest to similarity metric.

18. The method of claim 4, wherein the fingerprint is a Morgan fingerprint, extended connectivity fingerprint (ECFP), or other molecular fingerprint.

19. One or more non-transitory computer readable media storing instructions that in response to being executed by one or more processors, cause a computer system to perform operations, the operations comprising the method of claim 1.

20. A computer system comprising:
one or more processors; and
one or more non-transitory computer readable media storing instructions that in response to being executed by the one or more processors, cause the computer system to perform operations, the operations comprising the method of claim 1.
